(19) United States
(12) Patent Application Publication
(10) Pub. No.: US 2012/0004273 A1
(43) Pub. Date: Jan. 5, 2012

(54) GLYT1 TRANSPORTER INHIBITORS AND USES THEREOF IN TREATMENT OF NEUROLOGICAL AND NEUROPSYCHIATRIC DISORDERS

(76) Inventors: Nadia Mamoona Ahmad, Surrey (GB); Clive Leslie Branch, Essex (GB); Albert Andrzej Jaxa-Chamiec, London (GB); Justine Yeun Quai Lai, Essex (GB); David John Nash, Essex (GB)

(21) Appl. No.: 12/524,580

(22) PCT Filed: Jan. 30, 2008
(86) PCT No.: PCT/EP08/51084

§ 371 Date: Feb. 24, 2010

(30) Foreign Application Priority Data
Jan. 20, 2007 (GB) 0701986.2
Feb. 1, 2007 (GB) 0701977.1

(51) Int. Cl. A61K 31/4184; A61P 25/00; A61P 25/18; C07D 235/02

(52) U.S. Cl. 514/387; 548/300.7

(57) ABSTRACT

Disclosed herein are compounds of formula (I) and:

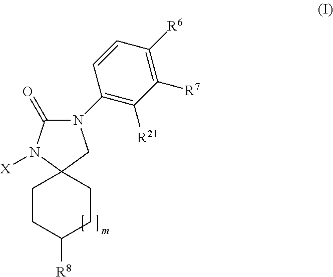

wherein:
X is Ar—C(O)CH(R²⁰)— or Ar₁—NHC(O)CH₂—; and Ar is:
naphthyl optionally substituted with one or more groups Y;
pyridyl optionally substituted with one or more groups Z; or
the group Ar₁

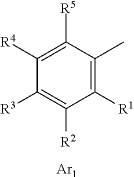

etc, these compounds being useful for treating neurological diseases such as schizophrenia, dementia or attention deficit disorder.

The present invention relates to compounds, processes for their preparation, pharmaceutical compositions and medicaments containing them and to their use in treating disorders mediated by GlyT1, including neurological and neuropsychiatric disorders, in particular psychoses, dementia or attention deficit disorder.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-Ia, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New End. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

It has now been found that a novel class of compounds inhibit GlyT1 transporters and are thus of potential utility in the treatment of certain neurological and neuropsychiatric disorders, including schizophrenia.

Thus, in the first aspect, there is provided a compound of formula (I) or a salt thereof:

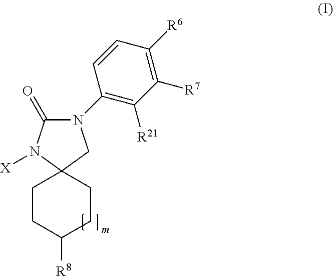

wherein:
X is selected from Ar—C(O)CH(R²⁰)— and Ar₁—NHC(O)CH₂—;
Ar is selected from:
naphthyl optionally substituted with one or more groups Y;
pyridyl optionally substituted with one or more groups Z; and
the group Ar₁

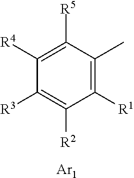

wherein:
Each Y is independently selected from C₁-C₄alkyl, C₁-C₄alkoxy, halo, haloC₁-C₄alkyl, haloC₁-C₄alkoxy, and cyano;
Each Z is independently selected from C₁-C₄alkyl, C₁-C₄alkoxy, halo, haloC₁-C₄alkyl, haloC₁-C₄alkoxy, and cyano;
R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{a}R^{b} (wherein R^{a} and R^{b} are independently selected from H and C₁-C₄alkyl, or R^{a} and R^{b}, together with the nitrogen atom to which they are attached, form a 4- to 7-membered ring) and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{c}R^{d} (wherein R^{c} and R^{d} are independently selected from H and C₁-C₄alkyl, or R^{c} and R^{d}, together with the nitrogen atom to which they are attached, form a 4- to 7-membered ring) and cyano;
R³ is selected from H, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{e}R^{f} (wherein R^{e} and R^{f} are independently selected from H and C₁-C₄alkyl, or R^{e} and R^{f}, together with the nitrogen atom to which they are attached, form a 4- to 7-membered ring) and cyano;
or R² and R³ together form a group selected from —O—CH₂—O— and —O—CH₂—CH₂—O—;
R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{g}R^{h} (wherein R^{g} and R^{h} are independently selected from H and C₁-C₄alkyl, or R^{g} and R^{h}, together with the nitrogen atom to which they are attached, form a 4- to 7-membered ring) and cyano;
R⁵ is selected from hydrogen, chloro, fluoro, C₁-C₄alkyl and CF₃;
R⁶ is selected from H, C₁-C₄alkyl, C₁-C₄alkoxy, haloC₁-C₄alkyl, haloC₁-C₄alkoxy, halo, cyano, C₁-C₄alkoxyC₁-C₄alkoxy, C₁-C₄alkoxyC₁-C₄alkyl, C₁-C₄alkylsulfonyl, C₁-C₄alkylthio, COR⁹ wherein R⁹ is hydrogen or C₁-C₄alkyl, CONRⁱR^{j} wherein Rⁱ and R^{j} are independently selected from hydrogen, C₁-C₄alkyl or, together with the nitrogen atom to which they are attached, form a 4, 5 or 6-membered ring, and CHR^{k}NR^{l}R^{m} wherein R^{k} is hydrogen or C₁-C₄alkyl and R^{l} and R^{m} are independently selected from hydrogen and C₁-C₄alkyl or R^{l} and R^{m}, together with the nitrogen atom to which they are attached, form a 4, 5 or 6-membered ring;
R⁷ is selected from H, C₁-C₄alkyl, C₁-C₄alkoxy, haloC₁-C₄alkyl, haloC₁-C₄alkoxy, halo, cyano, C₁-C₄alkoxyC₁-C₄alkyl and C₁-C₄alkoxyC₁-C₄alkoxy;
m is selected from 0, 1 and 2;
R⁸ is selected from hydrogen and C₁-C₄alkyl;
R²¹ is selected from H and fluoro; and
R²⁰ is selected from hydrogen and C₁-C₄alkyl.

The notations “C_{x-y}” and “Cₓ-C_{y}” are interchangeable.

As used herein, “a compound of the invention” means a compound of formula (I) or a salt, or solvate thereof.

It is to be understood that the present invention covers the compounds of formula (I) as the free base and as salts and solvates thereof, for example a pharmaceutically acceptable salt or solvate. It is to be further understood that references hereinafter to compounds of the invention or to compounds of formula (I) means a compound of formula (I) as the free base, or as a salt, or as a solvate.

As used herein, the term “C₁-C₄alkyl” refers to a straight or branched alkyl group of 1-4 carbon atoms in all isomeric forms. Examples include methyl, ethyl, propyl, isopropyl, butyl, isobutyl, sec-butyl and tert-butyl.

As used herein, the term “C₁-C₄alkoxy” refers to the group —O—C₁-C₄alkyl wherein C₁-C₄alkyl is as defined above.

As used herein, the term “C₁-C₄alkoxyC₁-C₄alkyl” refers to the group —(C₁-C₄alkyl)-O—(C₁-C₄alkyl), wherein C₁-C₄alkyl is as defined above.

As used herein, the term “C₁-C₄alkoxyC₁-C₄alkoxy” refers to the group —OC₁-C₄alkyl-O—C₁-C₄alkyl, wherein C₁-C₄alkyl is as defined above.

As used herein, the term “C₃-C₆cycloalkyl” refers to a cycloalkyl group consisting of from 3 to 6 carbon atoms, ie cyclopropane, cyclobutane, cyclopentane or cyclohexane.

As used herein, the terms “halogen” and its abbreviation “halo” refer to fluorine, chlorine, bromine, or iodine.

As used herein, the term “haloC₁-C₄alkyl” refers to a C₁-C₄alkyl group as defined above which is substituted with any number of fluorine, chlorine, bromine, or iodine atoms, including with mixtures of those atoms. A haloC₁-C₄alkyl group may, for example contain 1, 2 or 3 halogen atoms. For example, a haloC₁-C₄alkyl group may have all hydrogen atoms replaced with halogen atoms. Examples of haloC₁-C₄alkyl groups include, but are not limited to, fluoromethyl, difluoromethyl and trifluoromethyl.

As used herein, the term “haloC₁-C₄alkoxy” refers to a —C₁-C₄alkoxy group as defined above which is substituted with any number of fluorine, chlorine, bromine, or iodine atoms, including with mixtures of those atoms. A haloC₁-C₄alkoxy group may, for example contain 1, 2 or 3 halogen atoms. For example, a haloC₁-C₄alkoxy group may have all hydrogen atoms replaced with halogen atoms. Examples of haloC₁-C₄alkoxy groups include, but are not limited to, fluoromethyloxy, difluoromethyloxy and trifluoromethyloxy.

As used herein the term “cyano” refers to a group —CN.

As used herein, the term “C₁-C₄alkylsulfonyl” refers to a group —SO₂(C₁-C₄alkyl). An example is —SO₂CH₃.

As used herein, the term “C₁-C₄alkylthio” refers to a group —S—(C₁-C₄alkyl). An example is —SCH₃.

R^{a} and R^{b}, together with the nitrogen atom to which they are attached, may form a saturated 4- to 7-membered ring, ie an azetidinyl, pyrrolidinyl, piperidyl, or azepanyl group. Similarly, R^{c} and R^{d}, R^{e} and R^{f}, R^{g} and R^{h}, Rⁱ and R^{j}, and R^{l} and R^{m} may form such a group within the definition of formula (I) above.

In one embodiment X is Ar—C(O)CH(R²⁰)—

In one embodiment X is Ar₁—NHC(O)CH₂—;

In one embodiment R¹ is selected from H, C₁-C₂alkyl, C₁-C₂alkoxy, halo, haloC₁-C₂alkyl, haloC₁-C₂alkoxy, C₁-C₂alkylthio, C₁-C₂alkylsulfonyl, C₁-C₂alkoxyC₁-C₂alkyl and cyano. In a further embodiment R¹ is H.

In one embodiment R² is selected from H, C₁-C₂alkyl, C₁-C₂alkoxy, halo, haloC₁-C₂alkyl, haloC₁-C₂alkoxy, C₁-C₂alkylthio, C₁-C₂alkylsulfonyl, C₁-C₂alkoxyC₁-C₂alkyl and cyano. In one embodiment R² is H or halo. In a further embodiment R² is H or F. In one embodiment R² is H or C₁-C₂alkyl. In a further embodiment R² is H or methyl.

In one embodiment R³ is selected from H, C₁-C₂alkyl, C₁-C₂alkoxy, halo, haloC₁-C₂alkyl, haloC₁-C₂alkoxy, C₁-C₂alkylthio, C₁-C₂alkylsulfonyl, C₁-C₂alkoxyC₁-C₂alkyl and cyano; In a further embodiment R³ is H or halo. In a further embodiment R³ is H or F. In a further embodiment R³ is Cl.

In one embodiment R⁴ is selected from H, C₁-C₂alkyl, C₁-C₂alkoxy, halo, haloC₁-C₂alkyl, haloC₁-C₂alkoxy, C₁-C₂alkylthio, C₁-C₂alkylsulfonyl, C₁-C₂alkoxyC₁-C₂alkyl and cyano. In a further embodiment R⁴ is H or halo. In a further embodiment R⁴ is F. In a further embodiment R⁴ is H. In a further embodiment R⁴ is methyl.

In one embodiment R⁵ is H.

In one embodiment R⁶ is selected from H, C₁-C₂alkyl, C₁-C₂alkoxy, haloC₁-C₂alkyl, haloC₁-C₂alkoxy, halo, cyano, C₁-C₂alkoxyC₁-C₂alkoxy, C₁-C₂alkoxyC₁-C₂alkyl, C₁-C₂alkylsulfonyl, C₁-C₂alkylthio, and COR⁹ wherein R⁹ is hydrogen or C₁-C₂alkyl. In a further embodiment R⁶ is Cl, OCH₃ or CF₃.

In one embodiment R⁷ is selected from H, C₁-C₂alkyl, C₁-C₂alkoxy, haloC₁-C₂alkyl, haloC₁-C₂alkoxy, halo, cyano, C₁-C₂alkoxyC₁-C₂alkyl and C₁-C₂alkoxyC₁-C₂alkoxy. In a further embodiment R⁷ is Cl or H. In a further embodiment R⁷ is H or F

In one embodiment m is 1.

In one embodiment R⁸ is H.

In one embodiment R²⁰ is H.

In one embodiment there is provided a compound of formula (Ia) or a salt or solvate thereof:

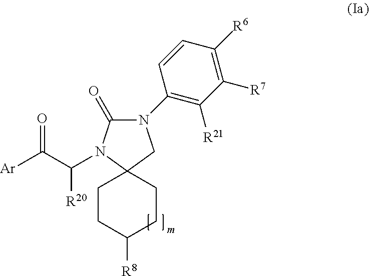

wherein:
Ar is selected from naphthyl optionally substituted with one or more groups Y, pyridyl optionally substituted with one or more groups Z, and the group

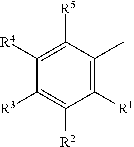

wherein:
Each Y is independently selected from C₁-C₄alkyl, C₁-C₄alkoxy, halo, haloC₁-C₄alkyl, haloC₁-C₄alkoxy, and cyano;
Each Z is independently selected from C₁-C₄alkyl, C₁-C₄alkoxy, halo, haloC₁-C₄alkyl, haloC₁-C₄alkoxy, and cyano;
R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxy C₁-C₄alkyl, CONR^{a}R^{b} (where R^{a} and R^{b} are independently selected from H and C₁-C₄alkyl or R^{a} and R^{b}, together with the nitrogen atom to which they are attached, form a 4- to 7-membered ring) and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxy C₁-C₄alkyl, CONR^{c}R^{d} (where R^{c} and R^{d} are independently selected from H and C₁-C₄alkyl or R^{c} and R^{d}, together with the nitrogen atom to which they are attached, form a 4- to 7-membered ring) and cyano;
R³ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{e}R^{f} (wherein R^{e} and R^{f} are independently selected from H and C₁-C₄alkyl, or R^{e} and R^{f}, together with the nitrogen atom to which they are attached, form a 4- to 7-membered ring) and cyano;
or R² and R³ together form a group selected from —O—CH₂—O— and —O—CH₂—CH₂—O—;
R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{g}R^{h} (wherein R^{g} and R^{h} are independently selected from H and C₁-C₄alkyl, or R^{g} and R^{h}, together with the nitrogen atom to which they are attached, form a 4- to 7-membered ring) and cyano;
R⁵ is selected from hydrogen, chloro, fluoro, C₁-C₄alkyl and CF₃;
R⁶ is selected from H, C₁-C₄alkyl, C₁-C₄alkoxy, haloC₁-C₄alkyl, haloC₁-C₄alkoxy, halo, cyano, C₁-C₄alkoxyC₁-C₄alkoxy; C₁₋₄alkoxyC₁₋₄alkyl, C₁₋₄alkylsulfonyl, C₁₋₄alkylthio, COR⁹ wherein R⁹ is hydrogen or C₁₋₄alkyl, CONRⁱR^{j} wherein Rⁱ and R^{j} are independently selected from hydrogen, C₁₋₄alkyl or, together with the nitrogen atom to which they are attached, form a 4, 5 or 6-membered ring, or CHR^{k}NR^{l}R^{m} wherein R^{k} is hydrogen or C₁₋₄alkyl and R^{l} and R^{m} are independently selected from hydrogen and C₁₋₄alkyl or R^{l} and R^{m}, together with the nitrogen atom to which they are attached, form a 4, 5 or 6-membered ring;
R⁷ is selected from H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;
m is selected from 0, 1 and 2;
R⁸ is selected from hydrogen and C₁-C₄alkyl;
R²¹ is selected from H or fluoro;
R²⁰ is selected from hydrogen and C₁-C₄alkyl.

In one embodiment, there is provided a compound of formula (Ib) or a salt or solvate thereof:

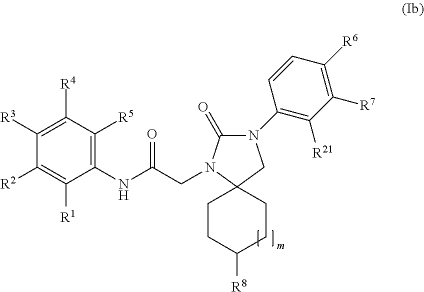

wherein:
R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{a}R^{b} (wherein R^{a} and R^{b} are independently selected from H and C₁-C₄alkyl, or R^{a} and R^{b}, together with the nitrogen atom to which they are attached, form a 4- to 7-membered ring) and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{c}R^{d} (wherein R^{c} and R^{d} are independently selected from H and C₁-C₄alkyl, or R^{c} and R^{d}, together with the nitrogen atom to which they are attached, form a 4- to 7-membered ring) and cyano;
R³ is selected from H, C₁₋₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{e}R^{f} (wherein R^{e} and R^{f} are independently selected from H and C₁-C₄alkyl, or R^{e} and R^{f}, together with the nitrogen atom to which they are attached, form a 4- to 7-membered ring) and cyano;
or R² and R³ together form a group selected from —O—CH₂—O— and —O—CH₂—CH₂—O—;
R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{g}R^{h} (wherein R^{g} and R^{h} are independently selected from H and C₁-C₄alkyl, or R^{g} and R^{h}, together with the nitrogen atom to which they are attached, form a 4- to 7-membered ring) and cyano;
R⁵ is selected from hydrogen, chloro, fluoro, C₁-C₄alkyl and CF₃;
R⁶ is selected from H, C₁-C₄alkyl, C₁-C₄alkoxy, haloC₁-C₄alkyl, haloC₁-C₄alkoxy, halo, cyano, C₁-C₄alkoxyC₁-C₄alkoxy; C₁₋₄alkoxyC₁₋₄alkyl, C₁₋₄alkylsulfonyl, C₁₋₄alkylthio, COR⁹ wherein R⁹ is hydrogen or C₁₋₄alkyl, CONRⁱR^{j} wherein Rⁱ and R^{j} are independently selected from hydrogen, C₁₋₄alkyl or, together with the nitrogen atom to which they are attached, form a 4, 5 or 6-membered ring, or CHR^{k}NR^{l}R^{m} wherein R^{k} is hydrogen or C₁₋₄alkyl and R′ and R^{m} are independently selected from hydrogen and C₁₋₄alkyl or R^{l} and R^{m}, together with the nitrogen atom to which they are attached, form a 4, 5 or 6-membered ring;
R⁷ is selected from H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;
m is selected from 0, 1 and 2;
R⁸ is selected from hydrogen and C₁-C₄alkyl;
R²¹ is selected from H or fluoro.

For the avoidance of doubt, the embodiments of any one feature of the compounds of the invention may be combined with any embodiment of another feature of compounds of the invention to create a further embodiment.

Examples of compounds of the invention include:

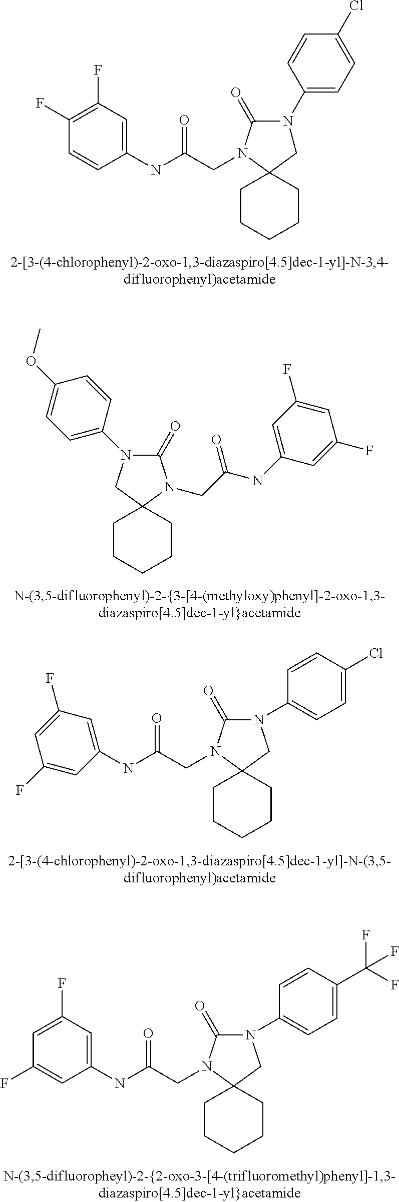

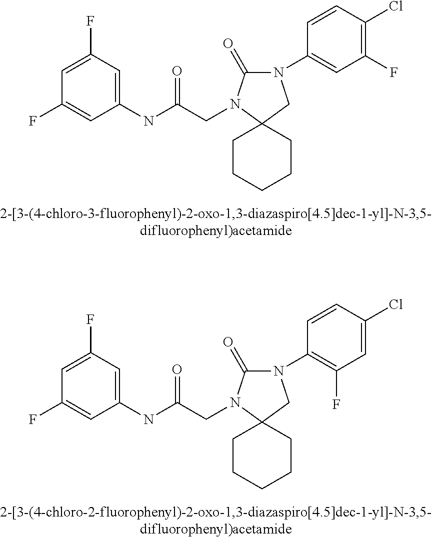

and salts and solvates thereof.

Further examples include:

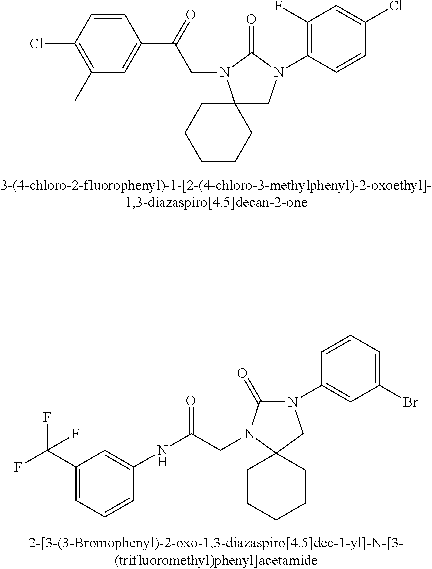

and salts and solvates thereof.

In an embodiment there is provided a compound of formula (I) as defined above or a pharmaceutically acceptable salt thereof.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1R)-(−)-10-camphorsulphonic, (1S)-(+)-10-camphorsulphonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic, and p-toluenesulfonic, acids. Salts having a non-pharmaceutically acceptable anion or cation are within the scope of the invention as useful intermediates for the preparation of pharmaceutically acceptable salts and/or for use in non-therapeutic, for example, in vitro, situations. The salts may have any suitable stoichiometry. For example, a salt may have 1:1 or 2:1 stoichiometry. Non-integral stoichiometry ratios are also possible.

Solvates of the compounds of formula (I) and solvates of the salts of the compounds of formula (I) are included within the scope of the present invention. As used herein, the term “solvate” refers to a complex of variable stoichiometry formed by a solute (in this invention, a compound of formula (I) or a salt thereof) and a solvent. Those skilled in the art of organic chemistry will appreciate that many organic compounds can form such complexes with solvents in which they are reacted or from which they are precipitated or crystallised. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water. Where the solvent used is water such a solvate may then also be referred to as a hydrate.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may be administered as prodrugs. Examples of pro-drug forms for certain compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Examples of prodrugs for certain compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Hereinafter, compounds of formula (I) (whether in solvated or unsolvated form) or their pharmaceutically acceptable salts (whether in solvated or unsolvated form) or prodrugs thereof defined in any aspect of the invention (except intermediate compounds in chemical processes) are referred to as “compounds of the invention”.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism) for instance when R⁸ in formula (I) is C₁-C₄alkyl. The individual stereoisomers (enantiomers and diastereoisomers) and mixtures of these are included within the scope of the present invention. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994). Likewise, it is understood that compounds of formula (I) may exist in tautomeric forms other than that shown in the formula and these are also included within the scope of the present invention.

In one embodiment, an optically pure enantiomer of a compound of the present invention is provided. The term “optically pure enantiomer” means that the compound contains greater than about 90% of the desired isomer by weight, such as greater than about 95% of the desired isomer by weight, or greater than about 99% of the desired isomer by weight, said weight percent based upon the total weight of the isomer(s) of the compound.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I).

Typical reaction routes for the preparation of a compound of formula (I) as hereinbefore defined, are shown below:

Compounds of formula (I) can be prepared by reacting a compound of formula (II) with a base, for example sodium hydride, in a suitable inert solvent, for example dimethylformamide, followed by treatment with a compound of formula (III) as shown in Scheme 1, where L is a leaving group such as chloro or bromo.

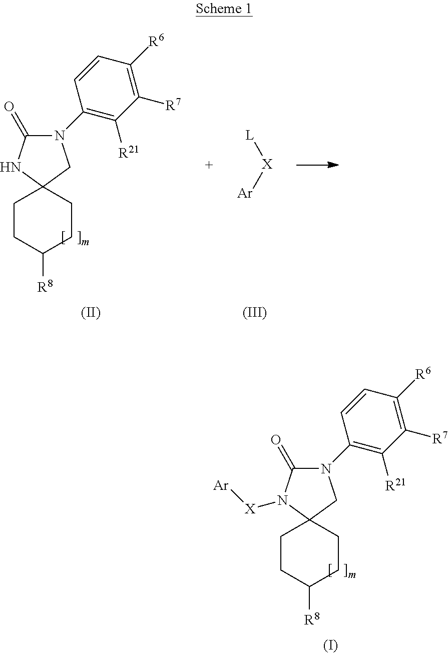

Compounds of formula (III) can be prepared by standard methods, for example as shown in Schemes 2A (X═—CR²⁰C(O)—) and 2B (X═—CH₂C(O)NH—).

For example an amide of formula (VA) may be reacted with a suitable organometallic reagent, for example methylmagnesium bromide or ethylmagnesium bromide, in an inert solvent, for example tetrahydrofuran, to afford the acetophenone (IVA) which can be converted to a compound of formula (IIIA), for example where L is a halogen, the acetophenone (IVA) can be halogenated, for example, with bromine, optionally in the presence of aqueous hydrogen bromide, in a solvent, such as acetic acid, to give a compound of formula (IIIA).

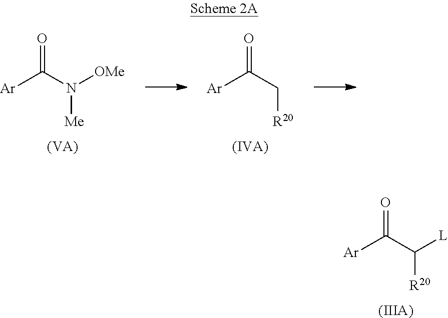

Alternatively, an aniline of formula (XVI) may be combined with an haloacetyl halide of formula (XVII) where X and X′ are halogen, for example chloroacetyl chloride or bromoacetyl chloride in an inert solvent, for example, dioxan and heated to give a compound of formula (IIIB).

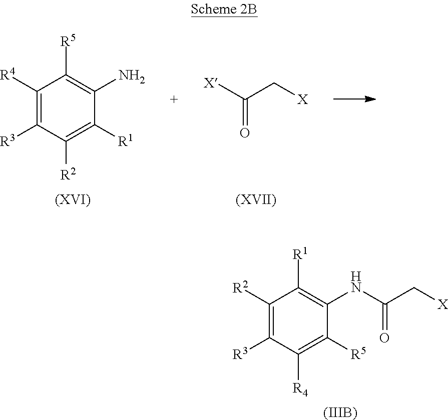

Compounds of formula (II) may be prepared by reduction of compounds of formula (V) using a reducing agent such as lithium aluminium hydride combined with aluminium trichloride in an inert solvent such as tetrahydrofuran at ambient or elevated temperature, preferably ambient temperature as shown for example in Scheme 3.

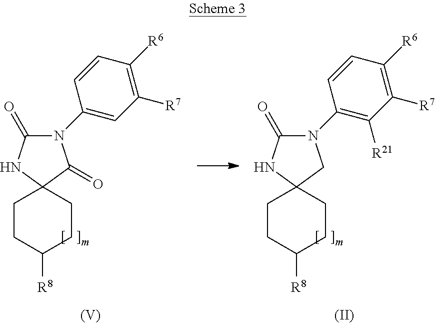

Compounds of formula (V) can be prepared by treating urea esters (VI) (shown as a methyl ester but could conveniently be any of C₁-C₆alkyl ester) with a base such as lithium hydroxide in aqueous tetrahydrofuran below ambient, ambient or elevated temperature, for example reflux as shown in scheme 4.

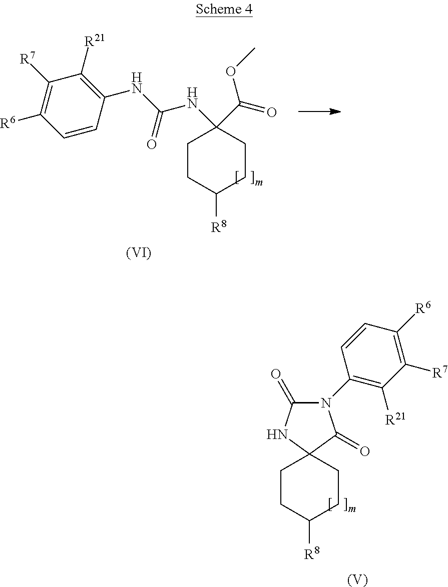

Urea esters of formula (VI) can be prepared in, for example, two steps, from the corresponding amino ester (VII) as in scheme 5.

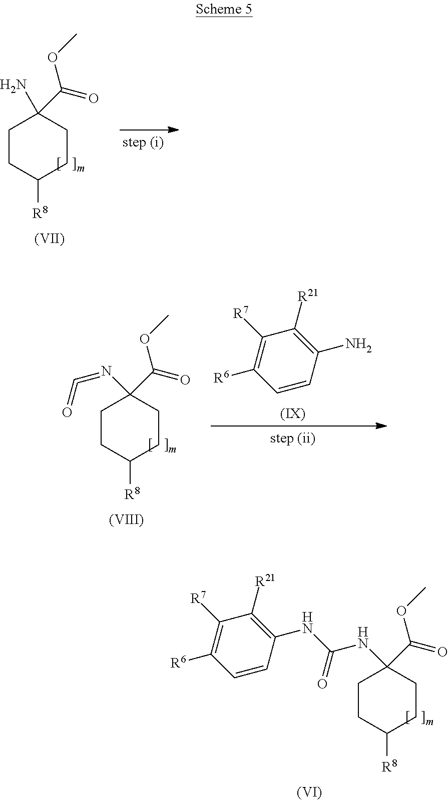

Treatment of amino esters (VII), step (i) with, for example, triphosgene or phosgene in an inert solvent such as dichloromethane or toluene in the presence of a base such as triethylamine, preferably at ambient temperature, generates isocyanates (VIII). Treatment of isocyanate (VIII), step (ii) with the appropriate substituted aniline (IX) generates the required urea esters of formula (VI).

Compounds of formula (II) can also be converted to compounds of formula (Ib) as shown in Scheme 6.

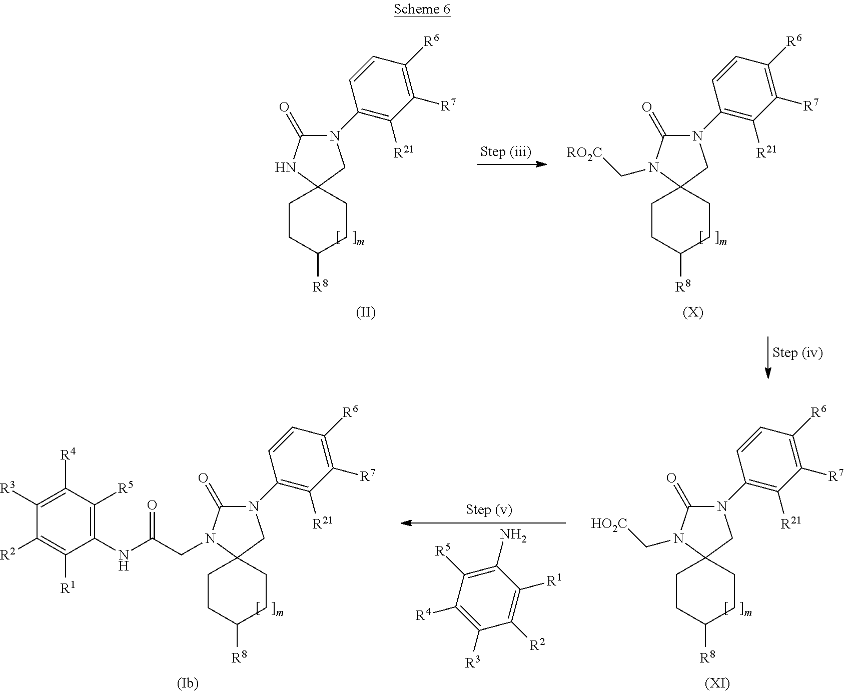

wherein R¹, R², R³, R⁴, R⁵, R⁶, R⁷, R⁸, R²¹ and m are as defined for compounds of formula (I).

Compounds of formula (X) can be prepared using standard methods from compounds of formula (II), step (iii), for example, by reaction with an appropriate haloester in the presence of a base, such as sodium hydride or potassium carbonate, in a suitable inert solvent, such as dimethylformamide, at room temperature or elevated temperature as appropriate.

Removal of the ester group R from compounds of formula (X) to afford the acids of formula (XI), step (iv), can be achieved by known methods, for example by use of a base, such as sodium hydroxide, in an inert solvent, such as aqueous methanol or aqueous ethanol, with or without heating as appropriate.

Compounds of formula (XI) can be converted to compounds of formula (I), step (v), by reaction with an aniline of formula (XVI) using a variety of methods known in the art. For example, the acylation step (v) can be achieved by reaction of the acid (XI) with an aniline of formula (XVI), in an inert solvent, such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), or O-(7-azabenzotriazol-1-yl)-1,1,3,3-tetramethyluronium hexafluoro phosphate (HATU). Alternatively, compounds of formula (XI) are converted to compounds of formula (XII)

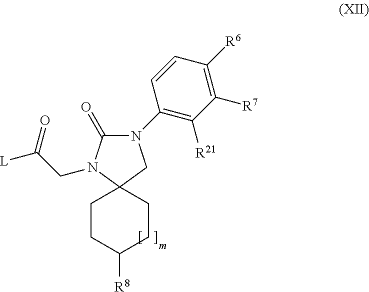

wherein R⁶, R⁷, R⁸, R²¹ and m are as defined in formula (I) and L represents a suitable leaving group. Examples of leaving groups include halogen, OC(═O)alkyl, OC(═O)O-alkyl and OSO₂Me. L may be halogen and acylation in step (vi) may be carried out in an inert solvent such as dichloromethane, in the presence of a base, such as triethylamine.

An additional approach to prepare compounds of formula (II) is outlined in scheme 7. Compounds of formula (XIII), which may be prepared according to methods described in U.S. Pat. No. 3,258,489 (1966), can be treated with a suitable reducing agent such as zinc powder in an acid such as acetic acid at, for example, ambient temperature to provide amines of formula (XIV). Treatment of compounds of formula (XIV) with a reagent such as phosgene in a solvent such as toluene or tetrahydrofuran or preferably a mixture of these solvents in the presence of a base such as triethylamine provides compounds of formula (II).

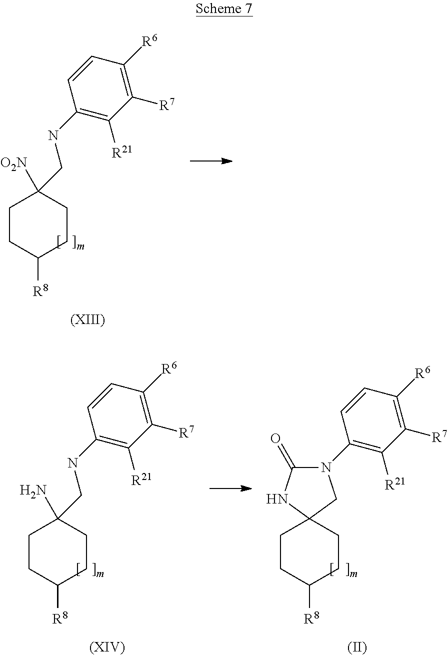

Alternatively compounds of formula (II) can be prepared according to scheme 8 and as described in Tetrahedron Lett. 2005.46.8555-8558 by treating compounds of formula (VII) (shown as a methyl ester but could conveniently be any of C₁-C₆alkyl ester) with an isocyanate of formula (XV) in an inert solvent such as dichloromethane, tetrahydrofuran or dimethylsulphoxide preferably in the presence of a base such as sodium carbonate. This reaction may be carried out at a range of temperature including ambient and elevated temperatures.

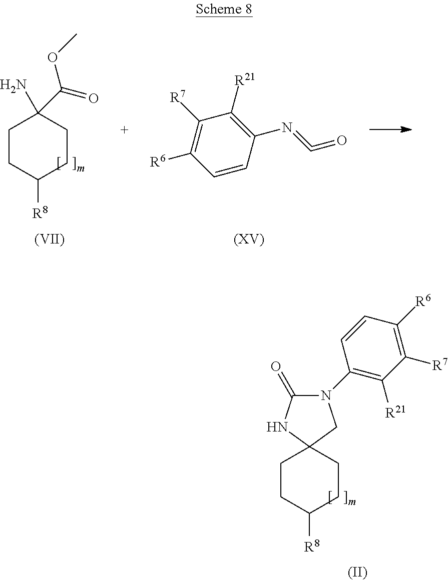

Within the scheme there is scope to convert a group R¹ into another group R¹ and similarly for groups R¹, R², R³, R⁴, R⁵R⁶, R⁷, R⁸, R²¹.

The compounds of the present invention inhibit the GlyT1 transporter, as measured by the assay below. Such compounds are therefore of potential utility for the treatment of certain neurological and neuropsychiatric disorders. The compounds may selectively inhibit the GlyT1 transporter over the GlyT2 transporter. Some compounds of the invention may have mixed GlyT1/GlyT2 activity.

The affinities of the compounds of this invention for the GlyT1 transporter can be determined by the following assay. In the assays used herein, the compounds of the present invention were not neccesarily from the same batch described above. The test compound made in one batch may have been combined with other batch(es) for the assay(s).

HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension and 5 μL of the cell/bead suspension transferred to each well of a 384-well white solid bottom plate (1,000 cells/well) containing 100 nL of test compounds. Substrate (5 μL) was added to each well [1:100 dilution of [³H]-glycine stock in assay buffer containing 2.5 μM glycine). Final DMSO concentration was 1% v/v. Data was collected using a Perkin Elmer Viewlux. plC₅₀ values were determined using ActivityBase.

Compounds are considered to have activity at the GlyT1 transporter if they have a plC₅₀ of 5.0 or above, conveniently a plC₅₀ at the GlyT1 transporter of greater than 6.0.

As used herein, the term “disorders mediated by GlyT1” refers to disorders that may be treated by the administration of a medicament that alters the activity of the GlyT1 transporter. The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury. Other disorders include Parkinson's disease, dyskinetic disorders, cognitive impairment, emesis, movement disorders, amnesia, circadian rhythm disorders, aggression and vertigo.

In one embodiment, the disorder mediated by GlyT1 to be treated by the use or method as hereinbefore described is a psychosis, including schizophrenia, dementia and attention deficit disorders. In one embodiment, the disorder is schizophrenia.

As used herein, the term “effective amount” means that amount of a drug or pharmaceutical agent that will elicit the biological or medical response of a tissue, system, animal or human that is being sought, for instance, by a researcher or clinician.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of the invention may be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of the invention may also be of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of the invention may also be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of the invention may also be of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of the invention may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds the invention may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of the invention may also be of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of the invention may also be of use in the treatment of Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of the invention may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention may also be of use for the treatment of cognition impairment which arises in association or as a result of other diseases such as schizophrenia, bipolar disorder, depression, other psychiatric disorders and psychotic conditions associated with cognitive impairment.

The compounds of the present invention may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of the invention may also be of use as anticonvulsants. The compounds of the invention are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of the invention as hereinbefore described or a salt thereof. Treatment of epilepsy may be carried out by the administration of a non-toxic anticonvulsant effective amount of a compound of the invention or a salt thereof.

The compounds of the invention may also be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

As used herein, the terms “treatment” and “treating” refer to the alleviation and/or cure of established symptoms as well as prophylaxis.

The invention thus provides compounds of formula (I) and salts thereof for use in therapy.

The invention also provides compounds of formula (I) and salts thereof for use in the treatment of a disorder mediated by GlyT1.

In a further aspect of the present invention, there is provided a method of treating disorders mediated by GlyT1 comprising administering a compound of formula (I) or a salt thereof.

In an further aspect of the present invention, there is provided the use of a compound of formula (I), or a salt thereof in the manufacture of a medicament for use in the treatment of disorders mediated by GlyT1.

In order to use a compound of the present invention in therapy, it will normally be formulated into a pharmaceutical composition in accordance with standard pharmaceutical practice. The present invention also provides a pharmaceutical composition, which comprises a compound of formula (I) or a salt thereof, and at least one pharmaceutically acceptable excipient.

In a further aspect, the present invention provides a process for preparing a pharmaceutical composition, the process comprising mixing a compound of formula (I) or a salt thereof and at least one pharmaceutically acceptable excipient.

A pharmaceutical composition of the invention is usually adapted for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment, oral administration is provided.

Compositions suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Compositions suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Compositions suitable for parenteral administration typically comprise sterile aqueous solutions containing a predetermined concentration of the active compound; the solution may be isotonic with the blood of the intended recipient. Such solutions may be administered intravenously or by subcutaneous or intramuscular injection.

Compositions suitable for rectal administration may be provided as unit-dose suppositories comprising the active ingredient and one or more solid carriers forming the suppository base, for example, cocoa butter.

Compositions suitable for topical or intranasal application include ointments, creams, lotions, pastes, gels, sprays, aerosols and oils. Suitable carriers for such compositions include petroleum jelly, lanolin, polyethylene glycols, alcohols, and combinations thereof.

The compositions of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 0.1 to about 1000 mg, for example about 0.5 mg to about 1000 mg, or about 1 mg to about 1000 mg, or about 5 mg to about 500 mg, or about 10 mg to about 100 mg of the active ingredient per unit dose, which could be administered, for example, 1 to 4 times per day.

The compounds of formula (I) and their salts thereof may also be suitable for combination with other therapeutic agents, such as typical and atypical antipsychotics. Thus, the present invention also provides:
i) a combination comprising a compound of formula (I) with one or more further therapeutic agents such as one or more antipsychotics;
ii) a pharmaceutical composition comprising a combination product as defined in i) above and at least one carrier, diluent or excipient;
iii) the use of a combination as defined in i) above in the manufacture of a medicament for treating or preventing a disease or condition caused by a reduction or imbalance in glutamate receptor function in a mammal;
iv) a combination as defined in i) above for use in treating or preventing a disease or condition caused by a reduction or imbalance in glutamate receptor function in a mammal;
v) a kit-of-parts for use in the treatment of a psychotic disorder comprising a first dosage form comprising a compound of the invention and one or more further dosage forms each comprising a antipsychotic agent for simultaneous therapeutic administration.
vi) a combination as defined in i) above for use in therapy;
vii) a method of treatment or prevention of a disease or condition caused by a reduction or imbalance in glutamate receptor function in a mammal comprising administering an effective amount of a combination as defined in i) above.

The combination therapies of the invention may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a salt thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, the compounds of formula (I) or a salt thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a salt thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a salt thereof to a patient receiving therapeutic administration of at least one antipsychotic agent. In a further aspect, the invention provides the use of compounds of formula (I) or a salt thereof in the manufacture of a medicament for adjunctive therapeutic administration for the treatment of a psychotic disorder in a patient receiving therapeutic administration of at least one antipsychotic agent. The invention further provides compounds of formula (I) or a salt thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a salt thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a salt thereof. The invention further provides at least one antipsychotic agent for adjunctive therapeutic administration for the treatment of a psychotic disorder in a patient receiving therapeutic administration of compounds of formula (I) or a salt thereof.

In a further aspect, the invention provides a method of treatment of a psychotic disorder by simultaneous therapeutic administration of compounds of formula (I) or a salt thereof in combination with at least one antipsychotic agent. The invention further provides the use of a combination of compounds of formula (I) or a salt thereof and at least one antipsychotic agent in the manufacture of a medicament for simultaneous therapeutic administration in the treatment of a psychotic disorder. The invention further provides the use of compounds of formula (I) or a salt thereof in the manufacture of a medicament for simultaneous therapeutic administration with at least one antipsychotic agent in the treatment of a psychotic disorder. The invention further provides compounds of formula (I) or a salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a salt thereof in the treatment of a psychotic disorder.

In further aspects, the invention provides a method of treatment of a psychotic disorder by simultaneous therapeutic administration of a pharmaceutical composition comprising compounds of formula (I) or a salt thereof and at least one mood stabilising or antimanic agent, a pharmaceutical composition comprising compounds of formula (I) or a salt thereof and at least one mood stabilising or antimanic agent, the use of a pharmaceutical composition comprising compounds of formula (I) or a salt thereof and at least one mood stabilising or antimanic agent in the manufacture of a medicament for the treatment of a psychotic disorder, and a pharmaceutical composition comprising compounds of formula (I) or a salt thereof and at least one mood stabilising or antimanic agent for use in the treatment of a psychotic disorder.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benziso-thiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs are as follows: clozapine (available under the tradename CLOZARIL®, from Mylan, Zenith Goldline, UDL, Novartis); olanzapine (available under the tradename ZYPREX®, from Lilly); ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK)); fluphenazine (available under the tradename PROLIXIN®, from Apothecon, Copley, Schering, Teva, and American Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman); perphenazine (available under the tradename TRILAFON®, from Schering); thioridazine (available under the tradename MELLARIL®, from Novartis, Roxane, HiTech, Teva, and Alpharma); molindone (available under the tradename MOBAN®, from Endo); and loxapine (available under the tradename LOXITANE®, from Watson). Furthermore, benperidol (Glianimon®), perazine (Taxilan®) or melperone (Eunerpan®) may be used. Other antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of formula (I) can be converted into further compounds of formula (I) using standard techniques. Salts may be prepared conventionally by reaction with the appropriate acid or acid derivative by the following non-limiting examples.

The starting material may not necessarily have been prepared from the batch detailed in the relevant Description. All quoted retention times are as measured using LC/MS (Liquid Chromatography/Mass Spectrometry). Where appropriate, these retention times were used as a guide for purification using mass-directed auto-preparation (MDAP), which refers to purification by HPLC, wherein fraction collection is triggered by detection of the programmed mass ion for the compound of interest.

Starting materials were obtained from commercial suppliers and used without further purification unless otherwise stated. Flash chromatography was carried out using pre-packed Isolute Flash™ or Biotage™ silica-gel columns as the stationary phase and analytical grade solvents as the eluent unless otherwise stated.

NMR spectra were obtained at between 294 and 296 K at 400 MHz frequency using either a Bruker™ DPX400 or AV400 machine and run as a dilute solution of CDCl₃ unless otherwise stated. All NMR spectra were referenced to tetramethylsilane (TMS δ_{H} 0, δ_{C} 0). All coupling constants are reported in hertz (Hz), and multiplicities are labelled s (singlet), bs (broad singlet), d (doublet), t (triplet), q (quartet), dd (doublet of doublets), dt (doublet of triplets) and m (multiplet).

Total ion current traces were obtained for electrospray positive and negative ionisation (ES+/ES−) and/or atmospheric pressure chemical positive and negative ionisation (AP+/AP−).

Unless otherwise stated, all compounds with chiral centre(s) are racemic.

Where reactions are described as having been carried out in a similar manner to earlier, more completely described reactions, the general reaction conditions used were essentially the same. Work up conditions used were of the types standard in the art, but may have been adapted from one reaction to another.

### ABBREVIATIONS

THF tetrahydrofuran
DCM dichloromethane
DMF dimethylformamide

### NMP N-methylpyrrolidinone

iPrOH isopropyl alcohol
IBX 1-hydroxy-1,2-benziodoxol-3(1H)-one-1-oxide
PE petroleum ether
EtOAc ethyl acetate
TLC thin layer chromatography
DMSO dimethylsulfoxide
EtOH ethanol
TFA trifluoroacetic acid
g grams
ml millilitres
mmol millimoles
r.t. room temperature
h hours
m minutes

Unless otherwise noted, all reagents were used without further purification. Further drying of, solvents was carried out in some cases. For example, Tetrahydrofuran (THF) was distilled from sodium under nitrogen and dichloromethane (DCM) was distilled from calcium hydride under nitrogen.

Analytical LC/MS Chromatography Conditions were Performed Either with:
Column: Waters Atlantis 50 mm×4.6 mm, 3 μm particle size
Mobile phase: A: 0.05% Formic acid+Water
B: Acetonitrile+0.05% Formic acid
   Gradient: 5-min runtime: 3% B to 97% B over 4 min
   Flow rate: 3 ml/min
   UV wavelength range: 220-330 nm

### Temperature: 30° C.

### Or

Column: Supelcosil ABZ+Plus 33 mm×4.6 mm, 3 μm particle size
Mobile phase: A: 10%[CH₃CN+0.05% TFA]
B: 90%[CH₃CN+0.05% TFA]
   Gradient: 2.8-min runtime A:B over 2.2 min
   Flow rate: 0.9 ml/min

### Temperature: 45° C.

### Preparative HPLC Conditions:

Preparative HPLC refers to methods where the material was purified by High Performance Liquid Chromatography on a Supelcosil ABZ+Plus 5 μm column (10 cm×21.2 mm). Eluting solvents were: water (containing 0.1% TFA) (A) and acetonitrile (containing 0.1% TFA) (B); 10 minute runtime with a gradient elution of 30-85% B at a flow rate of 8 mL/min and UV detection at 254 nm.

### Mass Directed Auto-Purification System Chromatography Conditions:

### Method 1

Column: Waters Atlantis 19 mm×100 mm or 30 mm×100 mm, 5 μm particle size
Mobile phase: A: 0.1% Formic acid+Water
B: Acetonitrile+0.1% Formic acid
   Gradient: 13.5 min runtime with 10 min gradient dependant on analytical retention time
   Flow rate: 20 or 40 ml/min

### Method 2

Mass-directed HPLC refers to methods where the material was purified by HPLC comprised of the following apparatus: Waters 600 gradient pump, Waters 2767 inject/collector, Waters reagent manager, Micromass ZMD mass spectrometer, Gilson Aspec—waste collector, Gilson 115 post-fraction UV detector. Detection was by UV and fraction collection was triggered by observation of the programmed mass ion for the compound of interest. Software used was Micromass Masslynx version 4.0. The column used was typically a Supelco LCABZ++column whose dimensions were 20 mm internal diameter by 100 mm in length. The stationary phase particle size was 5 μm. Eluting solvents were: water+0.1% formic acid (solvent A) and acetonitrile:water 95:5+0.05% formic acid (Solvent B). There were five methods used depending on the analytical retention time of the compound of interest. Each had a 15-minute runtime, which comprised a 10-minute gradient followed by a 5-minute column flush and re-equilibration step; MDP 1.5-2.2=0-30%; MDP 2.0-2.8=5-30%; MDP 2.5-3.0=15-55% B; MDP 2.8-4.0=30-80% B; MDP 3.8-5.5=50-90% B. Flow rate of 20 mL/min.

In general, where purifications involve the use of MDAP, Method 1 is employed unless otherwise stated.

### General

### Description 1: 2-Bromo-N-(3,5-difluorophenyl)acetamide

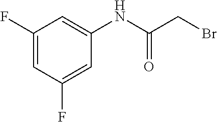

A mixture of 3,5-difluoroaniline (10.0 g; 77.45 mmol) and bromoacetyl bromide (6.73 ml; 77.45 mmol) in anhydrous dioxan (100 ml) was refluxed for 1.5 h, cooled to room temperature and diluted with water (400 ml) to afford a gum. The mother liquors were decanted and water added, followed by ethyl acetate. After stirring for 10 min the layers were separated and the organic layer dried and evaporated under reduced pressure. Recrystallisation from ethyl acetate-pentane afforded the title product as pale yellow crystals (6.5 g; 33%). ¹H NMR (CDCl₃) δ: 4.02 (2H, s), 6.60-6.65 (1H, m), 7.14-7.20 (2H, m), and 8.16 (1H, br s).

### Description 2: Methyl 1-isocyanatocyclohexanecarboxylate

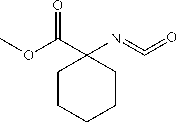

1-Aminocyclohexanecarboxyic acid (12.5 g, 87 mmol) was dissolved in 100 ml methanol and thionyl chloride (17.5 ml, 276 mmol) was added dropwise at −10° C. The mixture was refluxed for 4 hours and the solvent was removed in vacuo. The residue was slurried in NaHCO₃ solution and extracted with ethyl acetate. The solvent was distilled to yield a colorless oil, methyl 1-aminocyclohexanecarboxylate. Crude methyl 1-aminocyclohexanecarboxylate (2.05 g, 13.0 mmol) was dissolved in DCM (50 ml) and triethylamine (1.82 ml, 13.0 mmol) and triphosgene (1.28 g, 4.30 mmol) were added at 0° C. The mixture was stirred overnight at room temperature and the solvent was removed in vacuo. The residue was dissolved in ethyl acetate and the precipitate was filtered off. After evaporating, the crude product was used directly in Description 3 without purification.

### Description 3: Methyl 1-[({[4-(trifluoromethyl)phenyl]amino}carbonyl)amino]cyclohexanecarboxylate

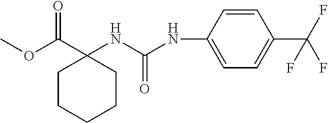

Methyl 1-isocyanatocyclohexanecarboxylate (1.83 g, 10.0 mmol, description 2) and 4-trifluoromethylaniline (1.61 g, 10.0 mmol) were dissolved in 10 ml CHCl₃ and stirred at room temperature overnight. The resulting white precipitate was filtered and washed with PE (10 ml). The solid was used without further purification in the next step.

### Description 4: 3-[4-(Trifluoromethyl)phenyl]-1,3-diazaspiro[4.5]decane-2,4-dione

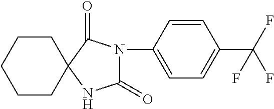

Methyl 1-[({[4-(trifluoromethyl)phenyl]amino}carbonyl)amino]cyclohexanecarboxylate (0.31 g, 1 mmol, description 3) was dissolved in 5 ml of THF and the mixture was cooled to 0° C. To this solution was added dropwise a solution of LiOH.H₂O (0.04 g, 1.0 mmol) in 2 ml water. After stirring for 30 min, PE (10 ml) was added to the mixture and the precipitate was filtered and washed with PE (10 ml). The solid was used without further purification in the next step.

### Description 5: 3-[4-(Trifluoromethyl)phenyl]-1,3-diazaspiro[4.5]decan-2-one

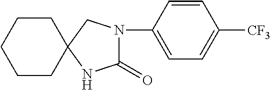

To a solution of LiAlH₄ (2.28 g, 20 mmol) in 20 ml of dry THF was added dropwise a solution of AlCl₃ (8.0 g, 60 mmol) in 100 ml dry THF under ice-cooling conditions. To this mixture was added slowly a solution of 3-[4-(trifluoromethyl)phenyl]-1,3-diazaspiro[4.5]decane-2,4-dione (6.24 g, 20 mmol, description 4) and AlCl₃ (2.66 g, 20 mmol) in 400 ml of dry THF at reflux temperature. After the addition was complete, the mixture was stirred for another 1 h under reflux. Then 200 ml Na₂CO₃ solution was added and the mixture was extracted with ether (4×100 ml). The ether phase was combined and distilled to dryness. The crude product was purified by column chromatography. LC/MS [m/z] calcd for C₁₅H₁₇F₃N₂O 298.31, found 299.3 (MH⁺).

### Description 6: Ethyl 1-aminocyclohexanecarboxylate hydrochloride

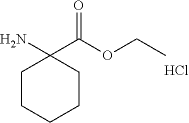

1-Aminocyclohexanecarboxylic acid (6.0 g, 41.90 mmol) and EtOH (150 ml) were mixed under nitrogen. The mixture was cooled below −10° C., then SOCl₂ (24 ml, 331 mmol) was added. The reaction was allowed to rise to ambient temperature and heated under reflux for 3 hours. Removal of the solvent under reduced pressure gave a white solid (9.0 g).

### Description 7: 4-Methoxyphenyl isocyanate

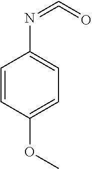

4-Methoxyaniline (10.0 g, 81.20 mmol) was dissolved in toluene and added dropwise to triphosgene (9.63 g, 32.50 mmol) under ice-bath. After the addition was complete, the reaction was warmed to ambient temperature and then heated to reflux for 3 hours under nitrogen. The contents were cooled to ambient temperature and filtered via silica gel, evaporated and the residue was used in the next preparation directly (˜10 g). LC/MS [m/z] calcd for C₈H₇NO₂ 149.05, found 148.96.

### Description 8: 3-[4-(Methyloxy)phenyl]-1,3-diazaspiro[4.5]decane-2,4-dione

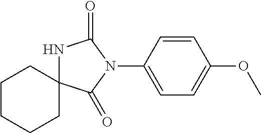

Ethyl 1-aminocyclohexanecarboxylate hydrochloride (0.80 g, 3.85 mmol, description 6) and Na₂CO₃ (0.82 g, 7.70 mmol) were dissolved in DMSO (20 ml) with stirring. After cooling to 0° C., a crude product of 4-methoxyphenyl isocyanate (0.63 g, 4.24 mmol, description 7) was added by syringe. The reaction was allowed to warm to ambient temperature and heated to 120° C., stirred for 2 hours and cooling to ambient temperature, quenched by addition of H₂O and the precipitate formed, collected by filtration and washed with water, and a white solid was obtained (0.90 g). LC/MS [m/z] calcd for C₁₅H₁₈N₂O₃ 274.13, found 275.1 (MH⁺).

### Description 9: 3-[4-(Methyloxy)phenyl]-1,3-diazaspiro[4.5]decan-2-one

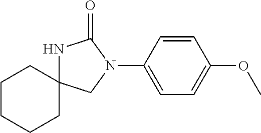

To a suspension of LiAlH₄ (1.74 g, 45.90 mmol) in dry THF (80 ml) was added AlCl₃ (6.12 g, 45.90 mmol) at 0° C. The contents were heated to reflux and stirred for 2 hrs under nitrogen, and then a mixture of 3-[4-(methyloxy)phenyl]-1,3-diazaspiro[4.5]decane-2,4-dione (4.20 g, 15.30 mmol, description 8) and AlCl₃ (2.04 g, 15.30 mmol) in dry THF (80 ml) added dropwise under reflux and stirred for 3 hrs. After cooling to 0° C., saturated Na₂CO₃ was added carefully, filtered and the filtrate was extracted with EtOAc twice, the organic phases were combined, dried (MgSO₄), filtered and evaporated, the residue was purified by recrystallization (PE/EtOAc), and a white solid was obtained (2.50 g, 62.80%). LC/MS [m/z] calcd for C₁₅H₂₀N₂O₂260.15, found 261.0 (MH⁺).

### Description 10: Methyl 1-aminocyclohexanecarboxylate

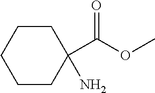

1-Amino-1-cyclohexane carboxylic acid (7.5 g, 52 mmol) was dissolved in methanol (120 ml) and thionyl chloride (10.5 ml, 1.166 mmol) was added dropwise at 0° C. The mixture was refluxed for 4 hrs and the solvent removed in vacuo. The residue was slurried in NaHCO₃ solvent and extracted with ethyl acetate to give a colourless oil (5.81 g, 71%). ¹HNMR (DMSO+D₂O): δ 4.12 (2H, s), 3.54 (3H, s), 1.70-1.77 (2H, m), 1.44-1.51 (2H, m), 1.22-1.33 (6H, m). LC/MS [m/z] calcd for C₈H₁₅NO₂ 157.11, found 157.1.

### Description 11: Methyl 1-isocyanatocyclohexanecarboxylate

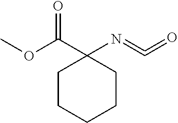

Triethylamine (5.16 ml, 44.4 mmol) and triphosgene (3.63 g) were added to a solution of methyl 1-aminocyclohexanecarboxylate (5.81 g, 37 mmol, description 10) in CH₂Cl₂ (140 ml), maintaining the temperature at 0° C. The mixture was then warmed to room temperature and allowed to stir overnight. TLC showed that all the starting materials were consumed. The solvent was removed by rotary evaporation under reduced pressure and the residue was dissolved in ethyl acetate and the precipitate was filtered off. After evaporation, methyl 1-isocyanatocyclohexanecarboxylate (5.42 g, 30 mmol) was obtained (yield: 80%). LC/MS [m/z] calcd for C₆H₁₃NO₃ 183.09, found 183.1.

### Description 12: Methyl 1-({[(4-chloro-2-fluorophenyl)amino]carbonyl}amino)cyclohexanecarboxylate

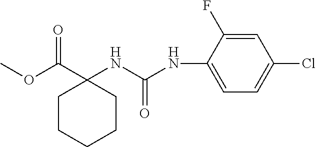

To a solution of 4-chloro-2-fluoroaniline (2.90 g, 20 mmol) in chloroform (30 ml) cooled to 0° C. with ice-water bath was added dropwise methyl 1-isocyanatocyclohexanecarboxylate (4.76 g, 26 mmol, description 11). The reaction mixture was stirred to form a clear solution and warmed to room temperature. After an additional 8 hours stirring, methyl 1-({[(4-chloro-2-fluorophenyl)amino]carbonyl}amino)cyclohexanecarboxylate (5.00 g, 15 mmol) was obtained by filtration and evaporation (yield: 75%). Mass Spectrum (ESI, LC/MS) calcd for C₁₅H₁₈ClFN₂O₃328.1, found 329.1 (MH⁺).

### Description 13: 3-(4-Chloro-2-fluorophenyl)-1,3-diazaspiro[4.5]decane-2,4-dione

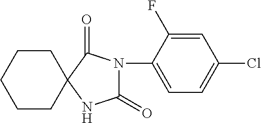

To a solution of methyl 1-({[(4-chloro-2-fluorophenyl)amino]carbonyl}amino)cyclohexanecarboxylate (5.0 g, 15 mmol, description 12) in 30 ml of THF was added 3 ml of 1M aqueous solution of LiOH at 0° C., then the reaction mixture was allowed to stir at room temperature for 5 hours. TLC showed that the starting material was consumed. The reaction mixture was filtered and evaporated to give 3-(4-chloro-2-fluorophenyl)-1,3-diazaspiro[4.5]decane-2,4-dione (3.33 g, 11.20 mmol), yield 75%. ¹H NMR (DMSO-d₆): δ 9.077 (1H, s), 7.62-7.64 (1H, m), 7.42-7.52 (2H, m), 1.22-1.7 (10H, m). LC/MS [m/z] calcd for C₁₄H₁₄ClFN₂O₂296.07, found 297.1 (MH⁺).

### Description 14: 3-(4-Chloro-2-fluorophenyl)-1,3-diazaspiro[4.5]decan-2-one

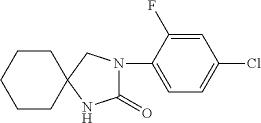

To a suspension of lithium aluminum hydride (1.58 g, 41.7 mmol) in THF (30 ml) was added a solution of AlCl₃ (5.56 g, 41.7 mmol) in dry THF (20 ml) at 0° C. with external cooling. The mixture was then refluxed for 1 hr and the rest portion of AlCl₃ (1.86 g, 13.9 mmol) and 3-(4-chloro-2-fluorophenyl)-1,3-diazaspiro[4.5]decane-2,4-dione (4.13 g, 13.9 mmol, description 13) dissolved in THF (40 ml), was slowly added dropwise maintaining refluxed over 2 hours. TLC showed that all the starting material was consumed. After cooling, the mixture was poured into water and basified with Na₂CO₃ (to pH 7-8), then the mixture was extracted with ethyl ether. The combined organics were washed with brine, dried over Na₂SO₄ and concentrated to get the crude product. After purification by chromatography, 3-(4-chloro-2-fluorophenyl)-1,3-diazaspiro[4.5]decan-2-one (0.63 g, 22.3 mmol) was obtained (yield: 16%). ¹H NMR (DMSO-d₆): δ 7.50-7.56 (2H, m), 7.40-7.46 (1H, m), 7.23-7.26 (1H, dd), 3.55 (2H, s), 1.34-1.62 (10H, m). LC/MS [m/z] calcd for C₁₄H₁₆ClFN₂O 282.09, found 283.1 (MH⁺).

### Description 15: 3-(4-Chlorophenyl)-1,3-diazaspiro[4.5]decane-2,4-dione

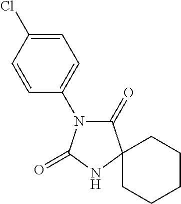

4-Chlorophenylisocyanate (8.0 g, 52.9 mmol) and sodium carbonate were added to DMSO and the mixture was stirred for 2 hrs. After cooling to 0° C. methyl 1-aminocyclohexanecarboxylate (8.0 g, 52.9 mmol, description 10) was added dropwise. After the addition was complete the mixture was heated to reflux for 2 hrs and then cooled to room temperature, when 60 ml of water was added, followed by filtration to give the product 3-(4-chlorophenyl)-1,3-diazaspiro[4.5]decane-2,4-dione (10.0 g, 55% yield). LC/MS [m/z] calcd for C₁₄H₁₅ClN₂O₂ 279.1 (MH⁺), found 279.0 (MH⁺).

### Description 16: 3-(4-Chlorophenyl)-1,3-diazaspiro[4.5]decan-2-one

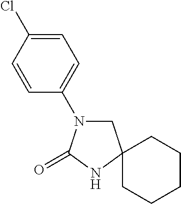

To the suspension of lithium aluminium hydride (1.2 g, 31.57 mmol) in THF was added AlCl₃ (12.0 g, 90 mmol), then the mixture was refluxed for 30 mins. A further portion of AlCl₃ (3.5 g, 26.3 mmol) was then added. 3-(4-Chlorophenyl)-1,3-diazaspiro[4.5]decane-2,4-dione (9.0 g, 32.5 mmol, description 15) was added dropwise at refluxing temperature and the mixture was refluxed for an additional hour. After cooling, the mixture was poured into ice water and extracted with ether twice. The combined ether layer was dried over Na₂SO₄ and concentrated to give the crude product, which was purified by column chromatography to give the pure product 3-(4-chlorophenyl)-1,3-diazaspiro[4.5]decan-2-one (1.5 g, 18% yield). ¹H NMR (d⁶-DMSO) δ: 1.45-1.67 (10H, m), 3.59 (2H, s), 5.14 (1H, s), 7.25-7.29 (2H, m), 7.48-7.51 (2H, m). LC/MS [m/z] calcd for C₁₄H₁₇ClN₂O 265.1 (MH⁺), found 264.9 (MH⁺).

### Description 17: Methyl 1-({[(4-chloro-3-fluorophenyl)amino]carbonyl}amino)cyclohexanecarboxylate

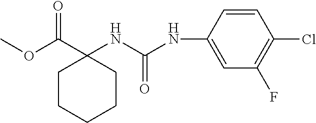

To a solution of methyl 1-isocyanatocyclohexanecarboxylate (4.0 g, 22 mmol, description 11) in chloroform (20 ml) cooled to 0° C. with ice-water bath, 4-chloro-3-fluoroaniline (3.0, 21 mmol) was added dropwise over 20 min, maintaining the temperature below 5° C. The reaction mixture was stirred at 0° C. for 30 min and at room temperature for 2 h. The precipitated solid was separated by filtration to give the title compound methyl 1-({[(4-chloro-3-fluorophenyl)amino]carbonyl}amino)cyclohexanecarboxylate (2.5 g).

### Description 18: 3-(4-Chloro-3-fluorophenyl)-1,3-diazaspiro[4.5]decane-2,4-dione

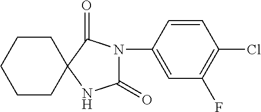

To a solution of methyl 1-({[(4-chloro-3-fluorophenyl)amino]carbonyl}amino)cyclohexanecarboxylate (3.5 g, 10.7 mmol, description 17) in THF was added 5 drops of 1M aqueous solution of LiOH at 0° C. The reaction mixture was allowed to stir at room temperature for 3 h. The reaction was filtered to give the desired product (4-chloro-3-fluorophenyl)-1,3-diazaspiro[4.5]decane-2,4-dione (3.2 g). LC/MS [m/z] calcd for C₁₄H₁₄ClFN₂O₂296.07, found 296(M⁺).

### Description 19: 3-(4-Chloro-3-fluorophenyl)-1,3-diazaspiro[4.5]decan-2-one

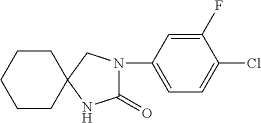

The title compound 3-(4-chloro-3-fluorophenyl)-1,3-diazaspiro[4.5]decan-2-one (1.0 g) was prepared according to the method of description 14 from 3-(4-chloro-3-fluorophenyl)-1,3-diazaspiro[4.5]decane-2,4-dione (3.2 g, 10.8 mmol). LC/MS [m/z] calcd for C₁₄H₁₆ClFN₂O 282.09, found 283.1 (MH⁺).

### Description 20: 2-Chloro-N-(3,4-difluorophenyl)acetamide

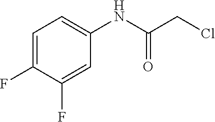

A mixture of 3,4-difluoroaniline (5.65 g; 50 mmol) and chloroacetyl chloride (6.46 g; 50 mmol) in dioxan (50 ml) was refluxed for 1 h, cooled to room temperature and concentrated under reduced pressure to a volume of 25 ml. Water was added to and the solid desired product separated by filtration and dried overnight (yield 9.41 g).

### Description 21: 1-({[(3-Bromophenyl)amino]carbonyl}amino)cyclohexanecarboxylic acid

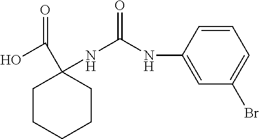

To a stirred solution of 1-aminocyclohexanecarboxylic acid (10 g, 69.8 mmol) was added a solution of sodium hydroxide (2.79 g, 69.8 mmol) in water (84 ml). The solution was cooled to 0° C., then 3-bromophenyl isocyanate (13.83 g, 69.8 mmol) was added portionwise over 15 minutes. The resulting solution was stirred at 0° C. for 3 hours then allowed to warm to room temperature. The reaction mixture was diluted with 1,4-dioxane (84 ml) and stirred at room temperature for 18 hours, then evaporated to dryness under reduced pressure to give the title compound as a cream solid (27.5 g). The LCMS showed a small impurity but the compound was taken through to the next stage with no further purification. Mass Spectrum (Electrospray LC/MS): Found 342 (MH⁺). C₁₄H₁₇⁷⁹BrN₂O₃ requires 341. Ret. time 2.48 min.

### Description 22: 3-(3-Bromophenyl)-1,3-diazaspiro[4.5]decane-2,4-dione

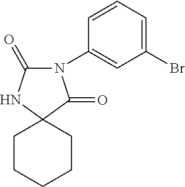

To a solution of 1-({[(3-bromophenyl)amino]carbonyl}amino)cyclohexanecarboxylic acid (D21) (27.5 g, 81 mmol) in ethanol (360 ml) was slowly added concentrated HCl acid (52 ml). The reaction mixture was heated at reflux whilst stirring for 6 hours. The mixture was allowed to cool to room temperature, and then cooled further using an ice bath for one hour. The white solid was filtered off and dried in the vacuum oven at 40° C. overnight to give the title compound (21.67 g). Mass Spectrum (Electrospray LC/MS): Found 325 (MH⁺). C₁₄H₁₅⁷⁹BrN₂O₂ requires 323. Ret. time 2.75 min.

### Description 23: 3-(3-Bromophenyl)-1,3-diazaspiro[4.5]decan-2-one

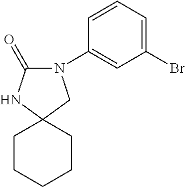

To a solution of lithium aluminium hydride (6.19 ml, 6.19 mmol) in 5 ml dry THF at 0° C. under argon was added aluminium chloride (0.825 g, 6.19 mmol) in 5 ml dry THF. The solution was stirred at 0° C. for 30 minutes before addition of a solution of 3-(3-bromophenyl)-1,3-diazaspiro[4.5]decane-2,4-dione (D22) (0.5 g, 1.547 mmol) in 5 ml dry THF. The resulting solution was then heated to 65° C. whilst stirring for 5 hours. The reaction mixture was allowed to cool to room temperature, and then cooled further to 0° C. 5M aqueous HCl acid was added dropwise until effervescence subsided. A further 5 ml was added, followed by 20 ml ethyl acetate. The mixture was stirred at room temperature for 48 hours, then the organic layer was separated and the aqueous layer extracted with 20 ml ethyl acetate. The organic layers were combined, dried with sodium sulfate and evaporated to dryness to give an orange oil, which was purified via Biotage (1:1 Hex/EtOAc, 25+M column). Fractions containing the desired product were combined and evaporated to dryness to give the title compound as a white solid (97 mg). Mass Spectrum (Electrospray LC/MS): Found 311 (MH⁺). C₁₄H₁₇⁷⁹BrN₂O requires 309. Ret. time 3.06 min.

### Description 24. 2-Bromo-N-[3-(trifluoromethyl)phenyl]acetamide

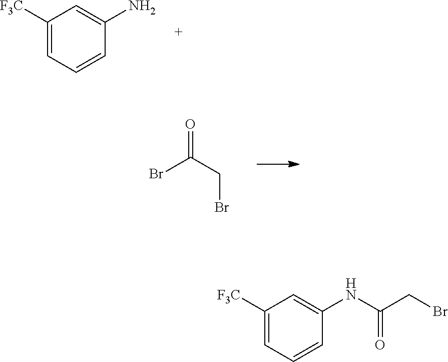

A stirred solution of 3-(trifluoromethyl)aniline (2.0 g, 0.012 mol) in dichloromethane (60 ml) at 10° C. under argon was treated dropwise over 5 minutes with bromoacetyl bromide (1.2 ml, 0.0137 mol). A white precipitate formed. This was allowed to warm to room temperature with good stirring over 1.5 hours, then treated with solid sodium hydrogen carbonate (1.65 g, 0.0196 mol) and stirred well for 40 minutes. The mixture was treated with water (100 ml), stirred well for 10 minutes, then the dichloromethane layer was isolated by passage through a phase separation cartridge and concentrated under reduced pressure to afford the title compound as a colourless oil (3.65 g, 100%). Mass Spectrum (Electrospray LC/MS): Found 282 (MH⁺). C₉H₇⁷⁹BrF₃NO requires 281. Ret. time 2.74 min.

¹H NMR δ (CDCl₃; 400 MHz): 4.05 (2H, s), 7.40-7.53 (2H, m), 7.76 (1H, d), 7.83 (1H, s), 8.24 (1H, br s).

### EXAMPLE 1

### N-(3,5-Difluorophenyl)-2-{2-oxo-3-[4-(trifluoromethyl)phenyl]-1,3-diazaspiro[4.5]dec-1-yl}acetamide

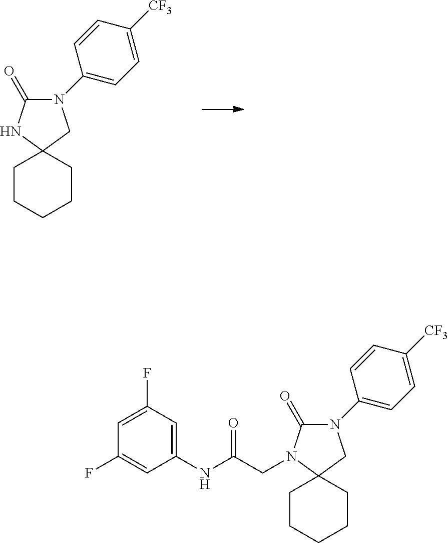

60% Sodium hydride in oil (15 mg; 0.369 mmol) was added to a stirred solution of 3-[4-(trifluoromethyl)phenyl]-1,3-diazaspiro[4.5]decan-2-one (100 mg; 0.335 mmol, description 5) in anhydrous DMF (3 ml) at room temperature under argon. After 10 minutes 2-bromo-N-(3,5-difluorophenyl)acetamide (D1) (92 mg; 0.369 mmol) was added in one portion. After 18 h further 60% sodium hydride in oil (15 mg; 0.369 mmol) was added, followed 30 minutes later by further 2-bromo-N-(3,5-difluorophenyl)acetamide (D1) (92 mg; 0.369 mmol). After stirring for a further 3 h saturated aqueous sodium hydrogen carbonate (150 ml) and brine (50 ml) were added and the mixture extracted with ethyl acetate (2×100 ml). The combined organics were dried (Na₂SO₄) and the solvent removed under reduced pressure. The residue was dissolved in DMSO to give 1.8 ml of solution and purified using mass directed auto-purification chromatography to give the title compound (45 mg; 28%). ¹H NMR (CDCl₃) δ: 1.15-1.26 (1H, m), 1.33-1.45 (2H, m), 1.50-1.80 (5H, m), 1.80-1.95 (2H, m), 3.73 (2H, s), 3.98 (2H, s), 6.49-6.56 (1H, m), 7.09-7.16 (2H, m), 7.61-7.63 (2H, m), 7.68-7.71 (2H, m), 9.15 (1H, s). Mass Spectrum (Electrospray LC/MS): Found 468 (MH⁺). C₂₃H₂₂F₅N₃O₂ requires 467. Ret. time 3.60 min.

The compounds in the table below were prepared using similar methods to that described in Example 1.

TABLE 1

| | | Mass spectrum | |
|---|---|---|---|
| | | (Electrospray | |
| | | LC/MS), API⁺ | |
| Ex | Structure | Ret. time (min) | Name |
| 2 | 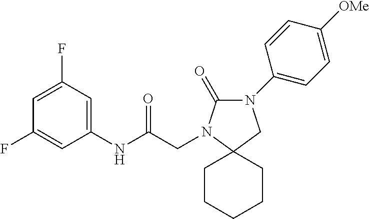 | Found 430 (MH⁺) C₂₃H₂₅F₂N₃O₃ requires 429; 3.41 | N-(3,5-difluorophenyl)-2-{3-[4- (methyloxy)phenyl]-2-oxo-1,3- diazaspiro[4.5]dec-1- yl}acetamide |
| 3 | 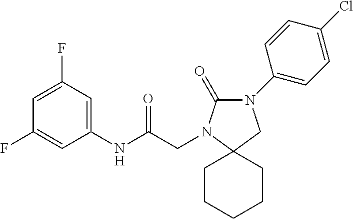 | Found 434 (MH⁺) C₂₂H₂₂³⁵ClF₂N₃O₂ requires 433; 3.63. | 2-[3-(4-chlorophenyl)-2-oxo-1,3- diazaspiro[4.5]dec-1-yl]-N-(3,5- difluorophenyl)acetamide |
| 4 | 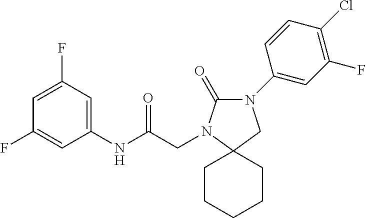 | Found 452 (MH⁺) C₂₂H₂₁³⁵ClF₃N₃O₂ requires 451; 3.63. | 2-[3-(4-chloro-3-fluorophenyl)-2- oxo-1,3-diazaspiro[4.5]dec-1-yl]- N-(3,5-difluorophenyl)acetamide |
| 5 | 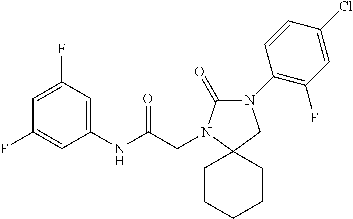 | Found 452 (MH⁺) C₂₂H₂₁³⁵ClF₃N₃O₂ requires 451; 3.56. | 2-[3-(4-chloro-2-fluorophenyl)-2- oxo-1,3-diazaspiro[4.5]dec-1-yl]- N-(3,5-difluorophenyl)acetamide |
| 6 | 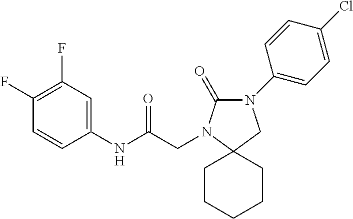 | Found 434 (MH⁺) C₂₂H₂₂³⁵ClF₂N₃O₂ requires 433. | 2-[3-(4-chlorophenyl)-2-oxo-1,3- diazaspiro[4.5]dec-1-yl]-N-(3,4- difluorophenyl)acetamide |

### EXAMPLE 7

### 3-(4-Chloro-2-fluorophenyl)-1-[2-(4-chloro-3-methylphenyl)-2-oxoethyl]-1,3-diazaspiro[4.5]decan-2-one

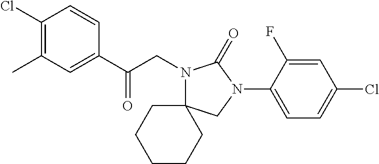

A solution of 3-(4-chloro-2-fluorophenyl)-1,3-diazaspiro[4.5]decan-2-one (D14) (198 mg, 0.7 mmol) in 4 ml DMF was treated under argon at room temperature with sodium hydride (60% suspension in mineral oil, 30.8 mg, 0.77 mmol) and stirred for 10 min. The orange mixture was still bubbling when the addition of a solution of 2-bromo-1-(4-chloro-3-methylphenyl)ethanone (173 mg, 0.7 mmol) in 7 ml DMF started. The addition was made over an hour with a syringe pump. The mixture was then allowed to stir for 1 hour after the end of addition. LC/MS showed a lot of unreacted urea, some desired product and some of the 2-bromo-1-(4-chloro-3-methylphenyl)ethanone left. Then 0.5 eq of sodium hydride was added and allowed to react for 30 min. LC/MS showed disappearance of the previously remaining 2-bromo-1-(4-chloro-3-methylphenyl)ethanone. Then 0.5 eq of the 2-bromo-1-(4-chloro-3-methylphenyl)ethanone was added and allowed to react for 1 h. LC/MS showed some 2-bromo-1-(4-chloro-3-methylphenyl)ethanone left and some more desired product. Then the mixture was heated at 60° C. overnight. LC/MS showed no 2-bromo-1-(4-chloro-3-methylphenyl)ethanone left, but still small amounts of the desired product. 0.5 eq of the 2-bromo-1-(4-chloro-3-methylphenyl)ethanone was added again and allowed to react at 80° C. for 4 hours. LC/MS showed no sensible evolution. Then 0.5 eq sodium hydride was added again and was allowed to react for 1 h. LC/MS showed no 2-bromo-1-(4-chloro-3-methylphenyl)ethanone left.

The mixture was concentrated under reduced pressure and the residue was purified by MDAP, but TLC of the resulting compound showed mixture of 2 major compounds with a few impurities. The compound was then purified by silica gel chromatography (EtOAc/hexane 5% to 60% in 10 column volumes) to give the desired compound 15 mg (0.033 mmol, ˜5% yield).

¹H NMR (CDCl₃) δ: 7.89 (1H, s), 7.78 (1H, dd), 7.57 (1H, t), 7.44 (1H, d), 7.12 (2H, m), 4.57 (2H, s), 3.75 (1H, s), 2.44 (3H, s), 1.78 (2H, t), 1.65 (1H, d), 1.46 (2H, td), 1.27 (2H, qt), 1.11 (1H, qt).

MS found (M+H)=449.2; C₂₃H₂₃³⁵Cl₂FN₂O₂ requires 448.

### EXAMPLE 8

### 2-[3-(3-Bromophenyl)-2-oxo-1,3-diazaspiro[4.5]dec-1-yl]-N-[3-(trifluoromethyl)phenyl]acetamide

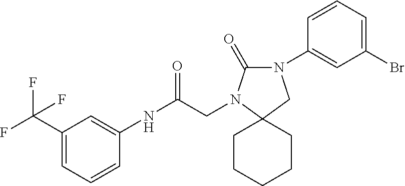

To a solution of 3-(3-bromophenyl)-1,3-diazaspiro[4.5]decan-2-one (D23) (44 mg, 0.142 mmol) in 2 ml DMF, at 0° C. was added sodium hydride (5.69 mg, 0.142 mmol). The solution was stirred at 0° C. for 30 minutes before addition of a solution of 2-bromo-N-[3-(trifluoromethyl)phenyl]acetamide (D24) (40.1 mg, 0.142 mmol) in 2 ml DMF using a syringe pump at 3 ml/hr. The solution was allowed to warm to room temperature and stirred for 18 hours. The reaction was quenched with methanol and the solution evaporated to dryness and the residue purified using mass directed auto-purification chromatography to give the title compound (6 mg). ¹H NMR (CDCl₃) δ: 0.95-1.25 (2H, m), 1.30-1.45 (2H, m), 1.65-1.75 (4H, m), 1.80-1.90 (2H, m), 3.70 (2H, s), 3.98 (2H, s), 7.20-7.26 (2H, m), 7.30-7.42 (2H, m), 7.55-7.57 (1H, m), 7.58-7.7 (2H, m), 7.85 (1H, s). Mass Spectrum (Electrospray LC/MS): Found 512 (MH⁺). C₂₃H₂₃⁷⁹BrF₃N₃O₂ requires 510. Ret. time 3.73 min.

1-27. (canceled)

28. A compound of formula (I) or a salt thereof: 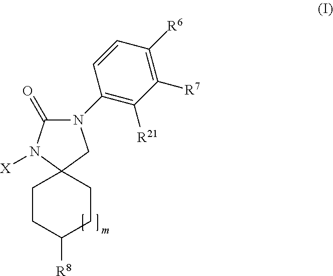
wherein:
X is Ar—C(O)CH(R²⁰)— or Ar₁—NHC(O)CH₂—;
Ar is:
naphthyl optionally substituted with one or more groups Y;
pyridyl optionally substituted with one or more groups Z; or
the group Ar₁ 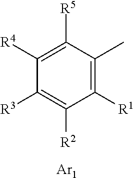
wherein:
each Y is independently C₁-C₄alkyl, C₁-C₄alkoxy, halo, haloC₁-C₄alkyl, haloC₁-C₄alkoxy, or cyano;
each Z is independently C₁-C₄alkyl, C₁-C₄alkoxy, halo, haloC₁-C₄alkyl, haloC₁-C₄alkoxy, or cyano;
R¹ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{a}R^{b} wherein R^{a} and R^{b} are independently H or C₁-C₄alkyl or R^{a} and R^{b} together with the nitrogen atom to which they are attached form a 4- to 7-membered ring or cyano;
R² is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{c}R^{d} wherein R^{c} and R^{d} are independently H or C₁-C₄alkyl, or R^{c} and R^{d}, together with the nitrogen atom to which they are attached, form a 4- to 7-membered ring or cyano;
R³ is H, C₁₋₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{e}R^{f} wherein R^{e} and R^{f} are independently H or C₁-C₄alkyl or R^{e} and R^{f}, together with the nitrogen atom to which they are attached form a 4- to 7-membered ring or cyano;
or R² and R³ together form the group —O—CH₂—O— or —O—CH₂—CH₂—O—;
R⁴ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{g}R^{h} wherein R^{g} and R^{h} are independently H or C₁-C₄alkyl or R^{g} and R^{h} together with the nitrogen atom to which they are attached form a 4- to 7-membered ring or cyano;
R⁵ is hydrogen, chloro, fluoro, C₁-C₄alkyl or CF₃;
R⁶ is H, C₁-C₄alkyl, C₁-C₄alkoxy, haloC₁-C₄alkyl, haloC₁-C₄alkoxy, halo, cyano, C₁-C₄alkoxyC₁-C₄alkoxy, C₁-C₄alkoxyC₁-C₄alkyl, C₁-C₄alkylsulfonyl, C₁-C₄alkylthio, COR⁹ wherein R⁹ is hydrogen or C₁-C₄alkyl, CONRⁱR^{j} wherein Rⁱ and R^{j} are independently hydrogen, C₁-C₄alkyl or together with the nitrogen atom to which they are attached form a 4, 5 or 6-membered ring, or CHR^{k}NR^{l}R^{m} wherein R^{k} is hydrogen or C₁-C₄alkyl and R^{l} and R^{m} are independently hydrogen or C₁-C₄alkyl or R^{l} and R^{m} together with the nitrogen atom to which they are attached form a 4, 5 or 6-membered ring;
R⁷ is H, C₁-C₄alkyl, C₁-C₄alkoxy, haloC₁-C₄alkyl, haloC₁-C₄alkoxy, halo, cyano, C₁-C₄alkoxyC₁-C₄alkyl or C₁-C₄alkoxyC₁-C₄alkoxy;
m is 0, 1 or 2;
R⁸ is hydrogen or C₁-C₄alkyl;
R²¹ is H or fluoro; and
R²⁰ is hydrogen or C₁-C₄alkyl.

29. A compound as claimed in claim 28 wherein X is Ar—C(O)CH(R²⁰)—

30. A compound as claimed in claim 28 wherein X is Ar₁—NHC(O)CH₂—;

31. A compound as claimed in claim 28 wherein R¹ is H.

32. A compound as claimed in claim 28 wherein R² is H or halo.

33. A compound as claimed in claim 28 wherein R² is H or F.

34. A compound as claimed in claim 28 wherein R² is H or methyl.

35. A compound as claimed in claim 28 wherein R³ is H or halo.

36. A compound as claimed in claim 28 wherein R³ is H, F or Cl.

37. A compound as claimed in claim 28 wherein R⁴ is H or halo or methyl.

38. A compound as claimed in claim 28 wherein R⁴ is F.

39. A compound as claimed in claim 28 wherein R⁵ is H.

40. A compound as claimed in claim 28 wherein R⁶ is Cl, OCH₃ or CF₃.

41. A compound as claimed in claim 28 wherein R⁷ is Cl or H or F.

42. A compound as claimed in claim 28 wherein m is 1.

43. A compound as claimed in claim 28 wherein R⁸ is H.

44. A compound as claimed in claim 28 wherein R²⁰ is H.

45. A compound as claimed in claim 28 which is:
2-[3-(4-chlorophenyl)-2-oxo-1,3-diazaspiro[4.5]dec-1-yl]-N-(3,4-difluorophenyl)acetamide;
N-(3,5-difluorophenyl)-2-{3-[4-(methyloxy)phenyl]-2-oxo-1,3-diazaspiro[4.5]dec-1-yl}acetamide;
2-[3-(4-chlorophenyl)-2-oxo-1,3-diazaspiro[4.5]dec-1-yl]-N-(3,5-difluorophenyl)acetamide;
N-(3,5-difluorophenyl)-2-{2-oxo-3-[4-(trifluoromethyl)phenyl]-1,3-diazaspiro[4.5]dec-1-yl}acetamide;
2-[3-(4-chloro-3-fluorophenyl)-2-oxo-1,3-diazaspiro[4.5]dec-1-yl]-N-(3,5-difluorophenyl)acetamide;
2-[3-(4-chloro-2-fluorophenyl)-2-oxo-1,3-diazaspiro[4.5]dec-1-yl]-N-(3,5-difluorophenyl)acetamide;
3-(4-chloro-2-fluorophenyl)-1-[2-(4-chloro-3-methylphenyl)-2-oxoethyl]-1,3-diazaspiro[4.5]decan-2-one;
2-[3-(3-Bromophenyl)-2-oxo-1,3-diazaspiro[4.5]dec-1-yl]-N-[3-(trifluoromethyl)phenyl]acetamide;
or a salt thereof.

46. A method for treating schizophrenia, dementia or attention deficit disorder comprising administering an effective amount of a compound of formula (I), or a pharmaceutically acceptable salt thereof, according to claim 28 to a patient in need thereof.

47. A pharmaceutical composition comprising a compound of formula (I), or a pharmaceutically acceptable salt thereof, as claimed in claim 28 and at least one pharmaceutically acceptable excipient.
